(19) United States
(12) Patent Application Publication
(10) Pub. No.: US 2014/0257049 A1
(43) Pub. Date: Sep. 11, 2014

(54) WEARABLE HEART MONITORING APPARATUS

(71) Applicant: Texas Instruments Incorporated, Dallas, TX (US)

(72) Inventors: Karthikeyan Soundarapandian, Plano, TX (US); Robert Burnham, Green Valley, AZ (US)

(73) Assignee: Texas Instruments Incorporated, Dallas, TX (US)

(21) Appl. No.: 14/190,834

(22) Filed: Feb. 26, 2014

Related U.S. Application Data
(60) Provisional application No. 61/769,088, filed on Feb. 25, 2013.

(51) Int. Cl. A61B 5/00; H05K 13/00; A61B 5/0245; A61B 5/021; A61B 5/024

(52) U.S. Cl. CPC A61B 5/681; A61B 5/021; A61B 5/02416; A61B 5/0245; A61B 5/6826; A61B 5/742; H05K 13/0023
USPC 600/301; 600/479; 29/592.1

(57) ABSTRACT

An apparatus mountable on a wearer's wrist includes a housing having at front portion and an opposite a back portion. The back portion is wearably positionable in contact with the wearer's wrist. The apparatus includes a PPG circuit for generating a PPG signal. The PPG circuit includes a light source and a photosensor on the housing back portion. The PPG signal may be used to continuously determine the wearer's a pulse rate. The PPG signal may also be used in combination with an ECG signal to determine the wearer's instantaneous blood pressure. The ECG signal may also be used to determine the wear's heart rate. The ECG signal may be generated with an electrode mounted on the back of the housing and another electrode mounted on another portion of the housing, such as the back or one or more of the sides.

### BACKGROUND

A photoplethysmogram (“PPG”) is an optically obtained plethysmogram, a volumetric measurement of an organ. A PPG may be obtained by using a device known as a “pulse oximeter,” which illuminates a person's skin with a light emitting diode (“LED”) and measures light absorption or reflection with a photodiode. The photodiode produces a PPG signal indicative of the measured light absorption or reflection. Changes in this PPG signal may be used to detect the pulse rate of the heart.

An electrocardiogram (“ECG”) is a test used to monitor the electrical activity of the heart. An ECG signal is produced by an electric current flowing between electrodes contacting different sites on a person's body. An ECG signal can be processed along with a PPG signal to determine a person's blood pressure.

During the past two decades a number of relatively inexpensive portable heart monitors have been developed, which operate through use of PPG signals and/or ECG signals. Such heart monitors enable a person to view and/or record his/her heart pulse rate and/or blood pressure during active exercise and without visiting a medical facility. Patents directed to heart monitors include the following U.S. Pat. No. 5,316,008 of Suga, et al., issued May 31, 1994; U.S. Pat. No. 5,865,755 of Golub, issued Feb. 2, 1999; U.S. Pat. No. 6,599,251 of Chen, et al., issued Jul. 29, 2003; U.S. Pat. No. 6,723,054 of Baruch, et al., issued Apr. 20, 2004; and U.S. Pat. No. 7,993,275 of Banet, et al., issued Aug. 9, 2011, which are all hereby incorporated by reference for all that is disclosed therein.

### BRIEF DESCRIPTION OF THE DRAWINGS

FIG. 1 is a schematic front view of a wearable heart monitoring apparatus.

FIG. 2 is a schematic back view of the wearable heart monitoring apparatus of FIG. 1.

FIG. 3 is a schematic cross sectional view of the wearable heart monitoring apparatus of FIGS. 1 and 2 mounted on a user's wrist.

FIG. 4 is a block diagram of functional units of a circuit board and display assembly.

FIG. 5 is a flow chart of a method of making a heart monitor.

FIG. 6 is a flow chart of a method for a person to monitor at least one of his/her pulse rate and blood pressure.

### DETAILED DESCRIPTION

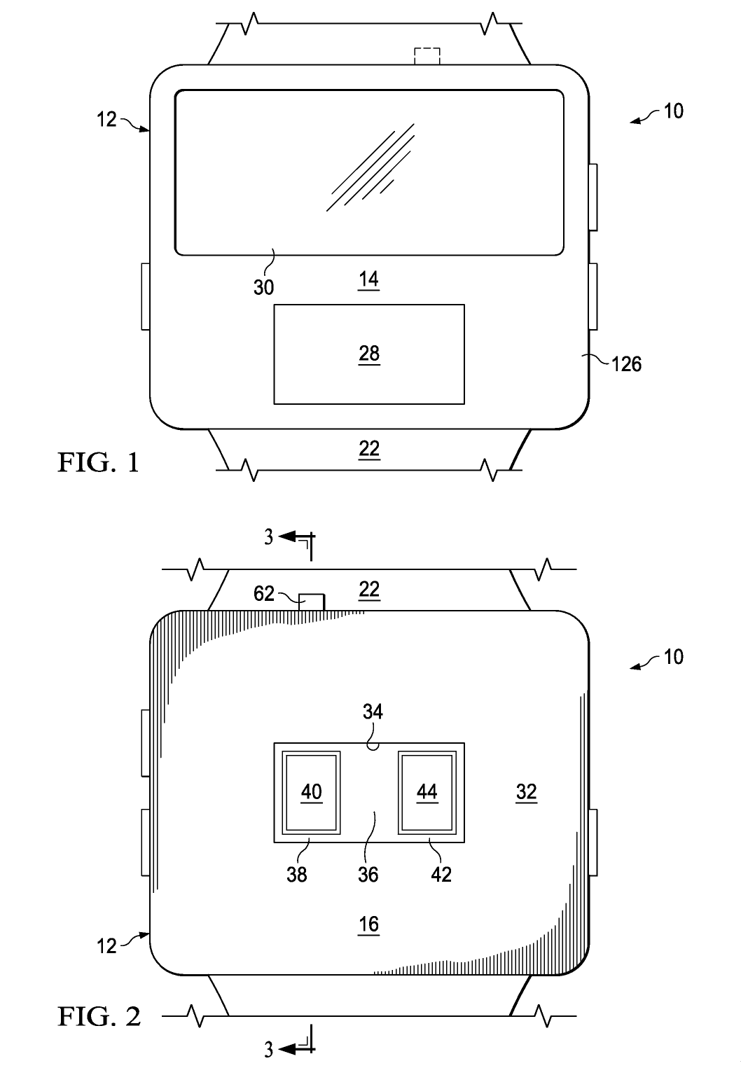

With reference to FIGS. 1-3, this specification, in general, discloses a heart monitoring apparatus 10 mountable on a human limb such as, for example a wrist 20. The apparatus includes a housing 12 having at front portion 14 and an opposite back portion 16. The back portion 16 is wearably positionable in contact with the wearer's limb. A PPG circuit for generating a PPG signal includes a light source, such as one or more LED's 44, on the housing back portion 16 and a photosensor, such as one or more photodiodes 40, also positioned on said housing back portion 16. The PPG circuit may be used to generate a pulse rate signal during the period that the apparatus 10 is worn. The apparatus 10, in one embodiment, also includes a ECG circuit. An ECG signal generated by the ECG circuit may be used in combination with the PPG signal to generate a blood pressure signal while a person touches a finger 60 to an electrode 32 positioned on the front surface 14. The ECG signal may also be processed to generate a heart rate signal. Having thus generally described an embodiment of a heart monitoring apparatus 10, various details thereof and related methods will now be described.

FIGS. 1-3 illustrate a wearable heart monitoring apparatus 10 having a generally wristwatch shaped housing 12. The housing has a front portion 14, FIG. 1, and an opposite back portion 16, FIG. 2. The back portion 16 is adapted to be positioned in touching engagement with the surface of a person's limb, for example it may be placed against his wrist 20. The apparatus 10 may be secured to the person's wrist 20 with a conventional watch band 22 or the like.

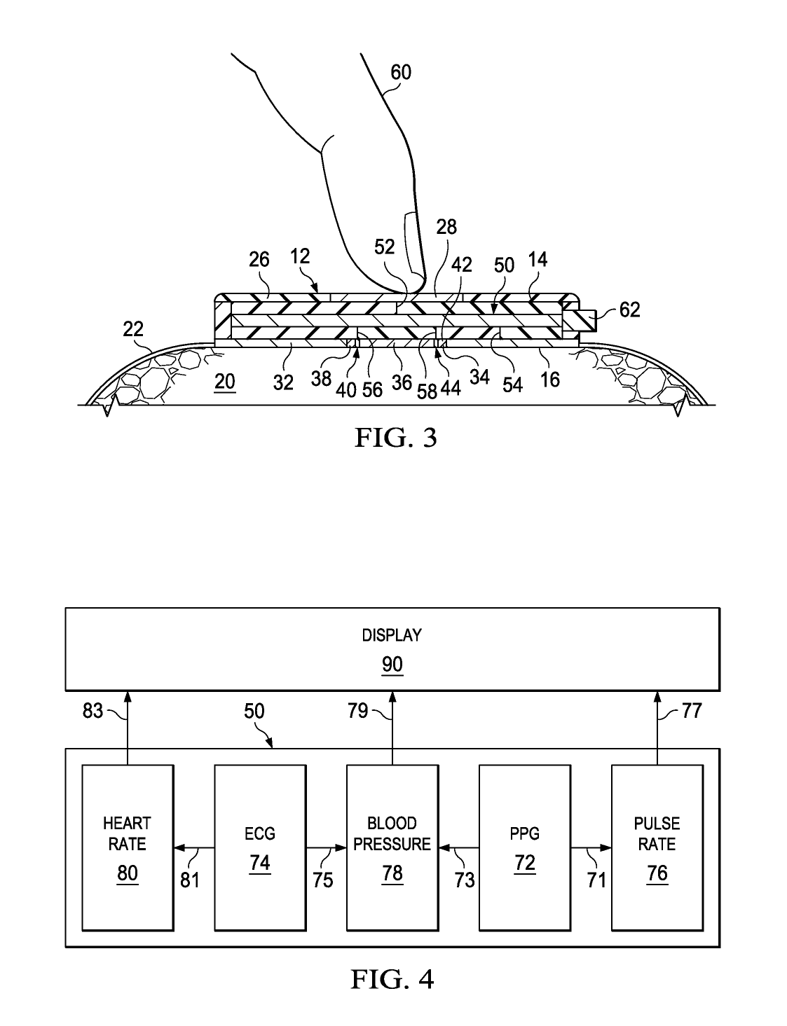

As illustrated by FIGS. 1 and 3, the front portion 14 of the housing 12 may be constructed primarily from a nonconductive material 26. A first electrode 28 of an ECG circuit, which is made from conductive material, is mounted on the front portion 14. An LCD display 30, FIG. 2, may also be provided on the front portion 14. The LCD display 30 may be adapted to display the user's heart rate, pulse rate or the user's blood pressure or any combination of the three. As used herein “heart rate” refers to the rate that the user's heart is beating. “Pulse rate refers to the rate at which the user's arteries pulse, i.e., go through expansion/contraction cycles. Although a user's heart rate and pulse rate are generally the same or about the same, there can be differences resulting from blockage in the arteries, etc.

As best illustrated in FIGS. 2 and 3, the back portion 16 of the housing 12 may be formed primarily from conductive material, which provides a second electrode 32 of the ECG circuit. An opening 34 is provided in the conductive material on the back portion 16. The opening 34 is divided by a partition 36 into a first section 38 having a photo diode 40 positioned therein and a second section 42 having an LED 44 positioned therein. The photo diode 40 and LED 44 are constructed and arranged such that the photo diode 40 only receives reflected light of the LED 44. The photo diode 40 generates a signal indicative of the reflected light from the person's skin that it receives. The photo diode 40 and LED 44 are part of a PPG circuit, which is further described below.

Referring to FIG. 3, a circuit board 50 is positioned in the wristwatch shaped housing 12 between the front portion 14 and the back portion 16. A first conductive wire 52 connects the first electrode 28 to the circuit board 50. A second conductive wire 54 connects the second electrode 32 to the circuit board 50. A third conductive wire 56 connects the photo diode 40 to the circuit board 50, and a fourth conductive wire 58 connects the LED 44 to the circuit board 50.

As shown by FIG. 4, the photo diode 40 and the LED 44 and circuitry within the circuit board 50 connected thereto comprise PPG circuitry 72 that generates a signal 71 which may be processed by conventional pulse rate processing circuitry 76 to determine the user's pulse rate. In operation, the LED 44 illuminates an oppositely positioned portion of the user's wrist 20. The light from the LED returns from the person's skin to the photo diode 40, which generates a PPG signal 71 that may be conventionally processed in a manner known in the art to produce a signal 77 representative of the user's pulse rate. The photo diode 40, third conductive wire 56, LED 44, fourth conductive wire 58 and associated circuitry within the circuit board 50 constitute a PPG circuit 72. The generation of an PPG signal based on the amount of light reflected from human skin is known in the art, such is shown for example by U.S. Pat. No. 5,316,008 incorporated by reference above, and is thus not further described herein.

The first electrode 28, first conductive wire 52, second electrode 32, second conductive wire 54 and associated circuitry within the circuit board 50 provides an ECG signal 75 when a person's finger 60 engages the electrode 28 while the second electrode 32 engages the surface of the user's wrist 20. This circuitry 28, 32, 52, 54 and associated circuitry within the circuit board 50 that generate the ECG signal comprise the ECG circuitry 74, FIG. 4. The generation of an ECG signal based on contact with two electrodes engaged by different parts of a person's body is known in the art, such is shown for example by U.S. Pat. No. 5,316,008 incorporated by reference above, and is thus not further described herein.

In one mode of operation, a PPG signal 71 may be generated continuously in response to actuation of a control surface such as push button 62 situated on the side of the housing and connected to circuit board 50. In a second mode of operation, which may be initiated by pushing button 62 in a preselected pattern, an ECG signal 75 is generated when the second electrode 32 on the back 16 of the housing 12 is engaged with a person's wrist 20 and a finger 60 of the user's other hand is engaged with the first electrode 28. The first and second mode of operation may occur simultaneously, such as by proper actuation of button 62. As illustrated by FIG. 4, during the first mode of operation, a first PPG signal 71 is sent to PPG processing circuitry 76 that processes the PPG signal 71 and generates a pulse rate signal 77. The pulse rate signal 77 may be sent to a display assembly 90 that generates a display representative of the person's pulse rate. In alternative embodiments the pulse rate signal 77 is transmitted to a remote station (not shown) for storage or viewing. In another embodiment the pulse rate signal 77 is stored in memory (not shown) in the apparatus 10.

As further illustrated by FIG. 4, in another mode of operation a second PPG signal 73, which may be the same as the first pulse signal 71, is input to blood pressure processing circuitry 78. In this mode of operation, the ECG circuitry 74 also sends an ECG signal 75 to the blood pressure processing circuitry 78. The processing circuitry 78, in this mode of operation, processes both the PPG signal 73 and the ECG signal 75 to generate a signal 79 representative of the wearer's blood pressure. This blood pressure signal 79 may be sent to the display assembly 90, and/or is transmitted to a remote station, and/or is stored either remotely or in the apparatus 10. It is known in the art to process an ECG signal and a PPG signal together to obtain a person's blood pressure such as disclosed, for example, in U.S. Pat. No. 5,316,008 incorporated by reference above. The techniques described in that patent or other similar known techniques may be used to produce the blood pressure signal 79, and thus the specifics of the ECG circuitry 74 and processing circuitry 78 are not further described herein.

As also illustrated by FIG. 4, in a still another mode of operation, the ECG circuitry 74 sends an ECG signal 81 to heart rate processing circuitry 80. The ECG signal 81 may be identical to ECG signal 75. The heart rate processing circuitry 80, in this fourth mode of operation, processes the ECG signal 81 to generate a signal 83 representative of the wearer's heart rate. This heart rate signal 83 may be sent to the display assembly 90, and/or is transmitted to a remote station, and/or is stored either remotely or in the apparatus 10. It is known in the art to process an ECG signal to obtain a person's heart rate, and thus the specifics of the ECG circuitry 74 and heart rate processing circuitry 80 are not further described herein.

It will be understood from the above that the pulse rate of the person wearing the heart rate monitoring apparatus 10 may be continuously calculated and updated and displayed during the entire period that the person is wearing the apparatus 10. It will also be understood that the person's blood pressure may be detected and displayed when the person is wearing the heart monitoring apparatus 10 on one wrist and is touching the front electrode 28 with a finger of the other hand, since both the ECG signal and the PPG signal are needed to determine the user's blood pressure. Also, the person's heart rate may be displayed when the person is wearing the heart monitoring apparatus 10 on one wrist and is touching the front electrode 28 with a finger of the other hand. The ECG signal can be processed without the PPG signal to generate and display the person's heart rate. Of course all or any combination of the pulse rate, blood pressure and heart rate may be determined and displayed when the person is wearing the apparatus on one limb and is touching the front electrode with another body part.

Although use of the heart monitoring apparatus 10 on a person wrist 20 is described, the apparatus could be worn other places, such as an ankle or upper arm or other body part. Also, rather than touching the electrode 28 on the front 14 of the apparatus with a finger 60 as described, the electrode could be touched with some other body part, for example a toe, located sufficiently distant from the site where the device is being worn. Furthermore, the apparatus 10 could be used on an animal other than a human so long as the LED 44 and photo diode 40, when worn by the animal, engage and unobstructed region of the animal's skin and the animal is caused to touch the front electrode 28 with the exposed skin of another body part.

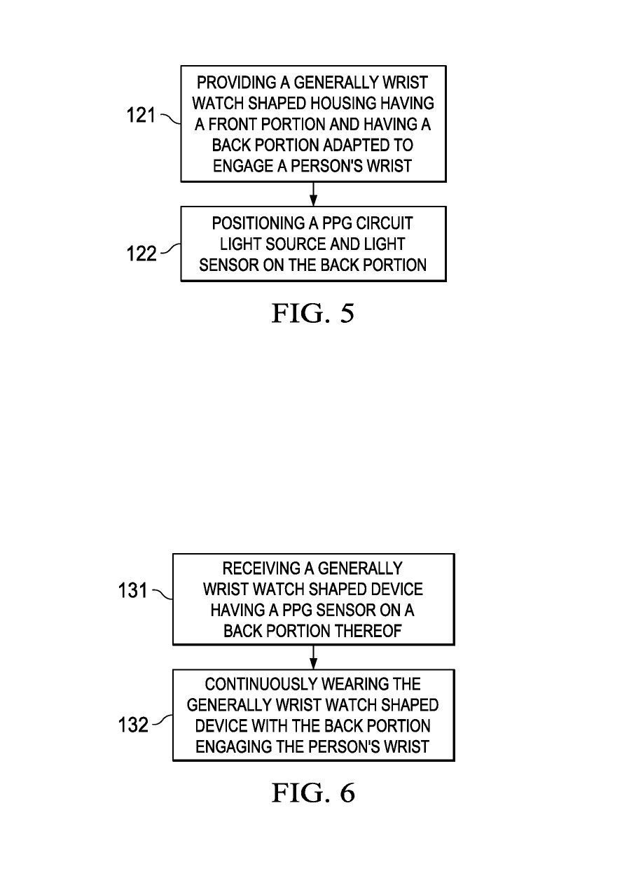

It will be appreciated from the above that, as shown by FIG. 5, a method of making a heart monitor has been described which comprises providing a generally wristwatch shaped housing having a front portion and having a back portion adapted to engage a person's wrist, block 121. The method also comprises positioning a PPG circuit light source and light sensor on the back portion, as illustrated in block 122.

It will also be appreciated from the above, that a method for a person to monitor at least one of his/her pulse rate and blood pressure may comprise, as shown in FIG. 6, block 131, receiving a generally wristwatch shaped device having a PPG photo sensor on a back portion thereof. The method may further comprise, as shown at block 132, continuously wearing the generally wristwatch shaped device with the back portion engaging the person's wrist.

Various embodiments of a wearable heart monitoring apparatus and methods of monitoring a person's pulse rate, heart rate and blood pressure have been described in detail herein. It is to be understood that the inventive concepts disclosed herein may be otherwise embodied, as will be obvious to those skilled in the art after reading this disclosure. It is intended that the appended claims be broadly construed to cover all such alternative embodiments, except as limited by the prior art.

What is claimed is:

1. An apparatus mountable on a human limb comprising:
a housing having at front portion and an opposite back portion, said back portion being wearably positionable in contact with a wearer's limb; and
a PPG circuit for generating a PPG signal including a light source on said housing back portion and a photosensor on said housing back portion.

2. The apparatus of claim 1 further comprising:
an ECG circuit for generating an ECG signal, said ECG circuit comprising:
a first electrode on said housing front portion; and
a second electrode on said housing back portion engageable with said wearer's limb.

3. The apparatus of claim 2, said first electrode being engageable with a wearer's finger.

4. The apparatus of claim 1 further comprising processing circuitry connected to said PPG circuit for continuously processing said PPG signal to generate a signal indicative of a wearer's pulse rate.

5. The apparatus of claim 2 further comprising processing circuitry for processing said PPG signal and said ECG signal to generate a signal indicative of a wearer's blood pressure when he is touching said first electrode.

6. The apparatus of claim 2 further comprising processing circuitry connected to said ECG circuit for processing said ECG signal to generate a signal indicative of a wearer's heart rate.

7. The apparatus of claim 4 further comprising a display operably connected to said processing circuitry for displaying indicia indicative of a wearer's pulse rate

8. The apparatus of claim 5, further comprising a display connected to said processing circuitry adapted to display indicia indicative of a wearer's blood pressure.

9. The apparatus of claim 6, further comprising a display adapted to display indicia indicative of a wearer's heart rate.

10. The apparatus of claim 1, said housing comprising a generally wristwatch shaped housing attachable to a wearer's wrist with a wrist band.

11. The apparatus of claim 2, said housing comprising a generally wristwatch shaped housing attachable to a wearer's wrist with a wrist band.

12. A method of making a heart monitor comprising:
providing a generally wristwatch shaped housing having a front portion and having a back portion adapted to engage a wearer's limb;
positioning a PPG circuit light source and light sensor on the back portion.

13. The method of claim 12 comprising mounting processing circuitry in the housing adapted to process a signal from the PPG circuit to determine the pulse rate of a wearer wearing the wristwatch shaped housing.

14. The method of claim 13 comprising mounting ECG signal generating circuitry in the housing including a first ECG circuit electrode mounted on the back portion of the housing and a second ECG circuit electrode mounted on the front portion of the housing.

15. The method of claim 14 comprising mounting further signal processing circuitry in the housing adapted to process signals from the PPG circuit and the ECG circuit to determine the instantaneous blood pressure of the wearer.

16. The method of claim 14 comprising mounting further signal processing circuitry in the housing adapted to process a signal from the ECG circuit to determine the heart rate of the wearer.

17. The method of claim 2 comprising mounting a display on the front portion of the housing adapted to display at least one of the wearer's pulse rate, heart rate and blood pressure determined using at least one of the PPG signal and the ECG signal.

18. A method for a person to monitor at least one of his/her pulse rate, heart rate and blood pressure comprising:
receiving a generally wristwatch shaped device having a PPG photosensor on a back portion thereof; and
continuously wearing the generally wristwatch shaped device with the back portion engaging the person's wrist.

19. The method of claim 18 wherein said continuously wearing the device comprises wearing the device with a first ECG electrode on the back portion of the device engaged with the person's wrist and further comprising engaging a second ECG electrode on a front portion of the device with one of the user's fingers.

20. The method of claim 19 further comprising reading at least one of a pulse rate, heart rate and blood pressure on a display on the front portion of the generally wristwatch shaped device.
